(19) United States
(12) Patent Application Publication
(10) Pub. No.: US 2018/0271374 A1
(43) Pub. Date: Sep. 27, 2018

(54) CHARACTERIZING NEUROLOGICAL FUNCTION AND DISEASE

(71) Applicants: David Byron Douglas, Winter Park, FL (US); Robert E. Douglas, Winter Park, FL (US); Kathleen M. Douglas, Winter Park, FL (US); Pamela K. Douglas, Winter Park, FL (US)

(72) Inventors: David Byron Douglas, Winter Park, FL (US); Robert E. Douglas, Winter Park, FL (US); Kathleen M. Douglas, Winter Park, FL (US); Pamela K. Douglas, Winter Park, FL (US)

(21) Appl. No.: 15/898,280

(22) Filed: Feb. 16, 2018

Related U.S. Application Data
(60) Provisional application No. 62/477,121, filed on Mar. 27, 2017.

(51) Int. Cl. A61B 5/00; A61B 5/055

(52) U.S. Cl. CPC A61B 5/0042; A61B 5/0478; A61B 5/055; A61B 5/4094

(57) ABSTRACT

A technique for correlating electroencephalogram (EEG) with diffusion tensor imaging (DTI) and magnetoencephalography (MEG) to place probes in optimal locations and generate 4D maps of neuronal activity in the brain is presented. EEG and MEG probes may be positioned relative to tract clusters based on a volumetric isotropic sequence and a DTI sequence. Each probe may include multiple electrodes. The probes may be associated with a helmet on which probe position is automatically adjusted. A baseline map may be compared with a pathological state map to aide in characterizing neurological function and disorder.

### TECHNICAL FIELD

The subject matter of this disclosure is generally related to characterizing neurological function and disorder.

### BACKGROUND

Epilepsy and certain other neurological disorders are characterized by seizures. A seizure can manifest in a variety of ways including generalized convulsions. Treatment options for epilepsy include medical therapy and, if refractory to medications, surgical resection of the epileptogenic foci. The decision making process with regard to surgical treatment can be quite complex. A key step in the surgical pre-operative planning is accurately localizing the seizure focus, i.e., identifying the location in the brain from which the seizures are originating.

Two techniques that are commonly used to help localize the seizure focus are neuroimaging and electroencephalography (EEG) exams. The neuroimaging exam most commonly performed to identify possible source locations of seizures is a magnetic resonance imaging (MRI) scan. MRI scanners use magnetic fields, radio waves and field gradients to generate images of the brain. Sometimes MRI scans reveal multiple possible epileptogenic foci and it is difficult for the neurosurgeon to determine which one to surgically resect. EEG scanners measure voltage potential differences between scalp electrodes and a reference or ground electrode. Sometimes, EEG is not adequate at localizing the source of seizures and more invasive electrocorticogram (ECoG) exams are performed inside of the skull. However, even the more invasive ECoG exam does not always provide the precise location from which the seizures originate. Thus, there is a compelling need for an improved technique for localizing seizure focus.

### SUMMARY

All examples, aspects and features mentioned in this document can be combined in any technically possible way.

In accordance with an aspect a method of characterizing neurological function of a brain of an individual, comprises: performing an MRI scan of the brain that includes a volumetric isotropic sequence and a diffusion tensor imaging (DTI) sequence to generate connectivity maps; establishing a common coordinate system for the connectivity maps and external features of the individual; positioning electroencephalography (EEG) probes based on the connectivity maps using the common coordinate system; performing an EEG scan during performance of prescribed tasks to generate EEG images; and generating 4D maps of neuronal activity from the EEG images and the connectivity maps. Some implementations comprise positioning magnetoencephalography (MEG) probes based on the connectivity maps using the common coordinate system. Some implementations comprise performing a MEG scan during performance of prescribed tasks to generate MEG images. Some implementations comprise generating the 4D maps of neuronal activity from the MEG images, the EEG images and the connectivity maps. Some implementations, wherein the brain is within a skull, and wherein using the connectivity maps to position the EEG probes, comprise positioning individual EEG probes proximate to locations where clusters of tracts are in close proximity to the skull. Some implementations comprise generating 4D maps of neuronal activity at a later time, thereby providing temporally distinct 4D maps of neuronal activity. Some implementations comprise performing a comparison of the temporally distinct 4D maps of neuronal activity. Some implementations comprise performing calibration before performing the EEG scan during performance of the prescribed tasks, the calibration comprising adjusting at least some EEG probe positions based on brain response to single sensor input. Some implementations comprise performing calibration before performing the EEG scan during performance of the prescribed tasks, the calibration comprising performing an EEG scan during a quiet period to document resting state brain activity. In some implementations the calibration comprises performing an EEG scan after the quiet period and while administering a series of stimuli. Some implementations comprise analyzing neuronal activity by converting EEG data to digital form and performing a Fourier transform on the digital EEG data to separate different brain waves. Some implementations comprise establishing nominal brain activity based on EEG data collected when no sensory stimuli are initiated. Some implementations comprise determining response time to input stimuli based on changes in brain wave activity at the EEG probes. Some implementations comprise preparing a time sequence of when EEG probes were triggered by the stimuli. Some implementations comprise performing a statistical analysis of response times where the stimuli are replicated. Some implementations comprise identifying a tract through correlation or coherence with the stimuli. Some implementations comprise inferring causality with respect to the identified tract by altering a ground or reference electrode for calculating voltage potential differences. Some implementations comprise registering DTI and EEG data to a canonical atlas that is built based on tract fiber lengths and thicknesses in relationship to EEG temporal and frequency content, direction, and sources. Some implementations comprise performing an analysis of speed of signals between EEG probes. Some implementations comprise performing a statistical analysis of cross stimuli response times and cross location of stimuli input locations. Some implementations comprise plotting in three dimensions all DTI threads and color coding voxels of thread groups. Some implementations comprise, using augmented reality, placing icons to denote locations of the EEG probes. Some implementations comprise representing direction of flow along the DTI threads, and time delays between sequential EEG probes.

In accordance with an aspect an apparatus comprises: a plurality of signal electrodes disposed in an array with insulating material between ones of the electrodes; and a reference electrode, wherein electrical output from each of the signal electrodes is measured relative to the reference electrode. In some implementations the array comprises 30 electrodes and the insulating material is circular with a 15 mm diameter. In some implementations the array comprises at least 1000 electrodes.

In accordance with an aspect an apparatus comprises: a helmet comprising: a net; and movable electroencephalography (EEG) probes. In some implementations the net conforms to a patient's head shape. In some implementations an automated system repositions the movable EEG probes based on a diffusion tensor imaging (DTI) scan.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is flow diagram that illustrates a technique for characterization of neurological function and disorder.

FIG. 2 is an isotropic T1 MRI depicting aspects of the positioning of EEG and MEG probes.

FIG. 3 is a volume rendered (VR) image of a DTI image superimposed on an isotropic T1 MRI image of the cingulate gyms.

FIG. 4 illustrates EEG leads superimposed on the DTI image with registration of the EEG and DTI onto a common coordinate system.

FIG. 5 is a flow diagram of data processing and analysis.

FIG. 6 illustrates a volume rendered image of a DTI image with tractography performed on the occipital radiations superimposed on an isotropic T1 MRI image to depict the arrival of signal from a flash of light in a dark room for the calculation of the rate of neuronal conduction.

FIG. 7 illustrates an overview of DTI, EEG and MEG on the same coordinate system.

FIG. 8 illustrates a process to visualize the DTI results, place electrode system components and time sequence brain activity recorded by the system electrode components.

FIGS. 9 and 10 illustrate the EEG electrode design; FIG. 10 represents section Z-Z of FIG. 9.

FIG. 11 illustrates a helmet with an array of high-density multiple electrode EEG probes.

FIG. 12 is a flow diagram that illustrates a calibration process for fine-tuning the location of electrode system components.

### DETAILED DESCRIPTION

U.S. Provisional Patent Application Ser. No. 62/477,121, entitled METHOD AND APPARATUS FOR CHARACTERIZING NEUROLOGICAL FUNCTION AND DISEASE, filed Mar. 27, 2017, is incorporated by reference.

Some aspects, features and implementations described herein may include machines such as computer devices, electronic components, and processes such as computer-implemented steps. It will be apparent to those of ordinary skill in the art that the computer-implemented steps may be stored as computer-executable instructions on a non-transitory computer-readable medium. Furthermore, it will be understood by those of ordinary skill in the art that the computer-executable instructions may be executed on a variety of tangible processor devices. For ease of exposition, not every step, device or component that may be part of a computer or data storage system is described herein. Those of ordinary skill in the art will recognize such steps, devices and components in view of the teachings of the present disclosure and the knowledge generally available to those of ordinary skill in the art. The corresponding machines and processes are therefore enabled and within the scope of the disclosure.

Gray matter contains most of the neuronal cell bodies of the brain, including specialized regions associated with functions such as muscle control, sensory perception, memory, emotions, speech, decision making, and self-control. White matter is composed of bundles of myelinated nerve cell projections called “axons” that interconnect different areas of gray matter by carrying nerve impulses between neurons. The human brain may contain 100 billion axons. EEG measurements may reflect the major communication structures of the brain as opposed to the neurons themselves. Anisotropic tissue, which can be imaged via diffusion tensor imaging (DTI), can conduct electricity in the same direction-specific manner in axons. DTI is a type of neuroimaging that can be performed on a Mill scanner to provide information about the brain's neuroanatomic connectome. DTI gives insight into the 3D architecture of the white matter of the brain. The inventors have recognized that DTI and EEG can be used together to improve localization of seizure focus.

A technique for characterization of neurological function and disorder may include use of DTI and EEG to generate a 4D electrical conduction map of the brain during a healthy state. The healthy state map can be compared with a corresponding 4D electrical conduction map generated during a pathological state. Other aspects of the technique may include optimal placement of EEG leads with guidance from the isotropic sequence and DTI imaging, and mapping the precise neural pathways for individual tasks. Benefits may include early detection of pathology, localization of pathology and mapping neural pathways of how a person thinks.

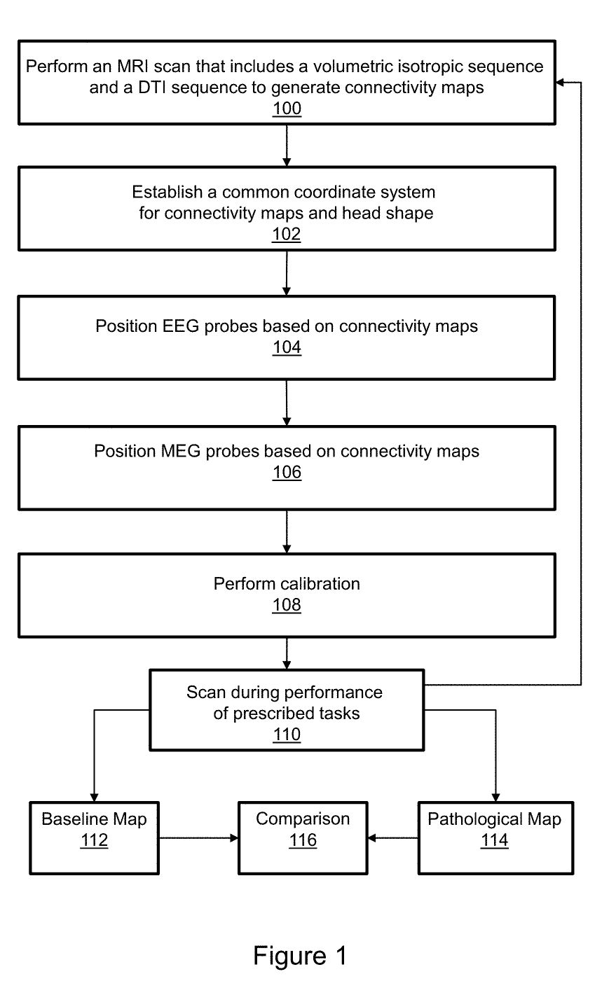

Referring now to FIG. 1, an initial step 100 of a technique for characterization of neurological function and disorder is to perform neuroimaging such as an MRI scan that includes a volumetric isotropic sequence and a DTI sequence. This will yield connectivity maps of the precise structure and directions of the axonal anatomy of the brain of an individual. The next step 102 is to establish a common coordinate system for the connectivity maps relative to the head of the individual. Specifically, a common coordinate system may be established for the person's head shape with reference to external features or landmarks such as nose, eyes, ears, skin markers, etc. As will be explained below, EEG scan images may be registered onto that common coordinate system with the DTI images in order to establish an anatomically enhanced EEG. The next step 104 is to position the EEG probes based on the connectivity maps using the common coordinate system. The EEG probes may be positioned based on gyral anatomy as indicated by the isotropic sequence and DTI imaging performed in step 100. Positioning based on gyral anatomy is described in greater detail below. The next step 106 is to position magnetoencephalography (MEG) probes based on the connectivity maps using the common coordinate system. MEG is a type of functional neuroimaging that assesses changes in magnetic fields induced by the changing electrical currents within the brain during neuronal activity. MEG would not necessarily have to be used, but it may serve to increase the accuracy of localization of the electrical activity along the axons within the brain. The next step 108 is a calibration process to adjust probe positions and determine metrics of neuronal activity and conduction. Some aspects of calibration may be performed in a dark, sound-proof room such that controlled stimuli could be administered to the individual. After a quiet period to document resting state brain activity, a series of stimuli may be provided while scanning (MRI, EEG, MEG and combinations thereof). For example and without limitation a point light source in the dark environment administered via virtual reality goggles; a single sound in the quiet environment; a tactile buzzer on finger; a simple math problem; facial recognition, etc. These stimuli would be replicated for both sides of the body. The next step 110 is to conduct scans (MRI, EEG, MEG or combinations thereof) during performance of prescribed tasks by the individual in the dark room. A wide variety of prescribed tasks might be utilized, possibly including but not limited to sensory, motor or cognitive tests such as language, memory, mathematics, problem solving or psychological testing. This would establish the individual's baseline (healthy state) map 112 of neurologic health and function. Such collected data would be analyzed as will be described in greater detail below and stored as a baseline “map” or “atlas” for use at a later time.

At some later time a follow-up analysis may be performed. The follow-up analysis may include performance of some or all of the baseline analysis steps 100 through 110 already described. For example and without limitation, the DTI imaging would not necessarily have to be repeated. The follow-up analysis would establish the individual's pathological (unhealthy state) map 114 of neurologic health and function. One purpose of the follow-up testing is to assess changes in the neurological health after a neurological insult (e.g., traumatic brain injury) or for changes in the neurological health in high risk populations (e.g., cancer patients) or to assess psychiatric treatment monitoring. This may include comparison of the baseline map with the pathological state map in step 116.

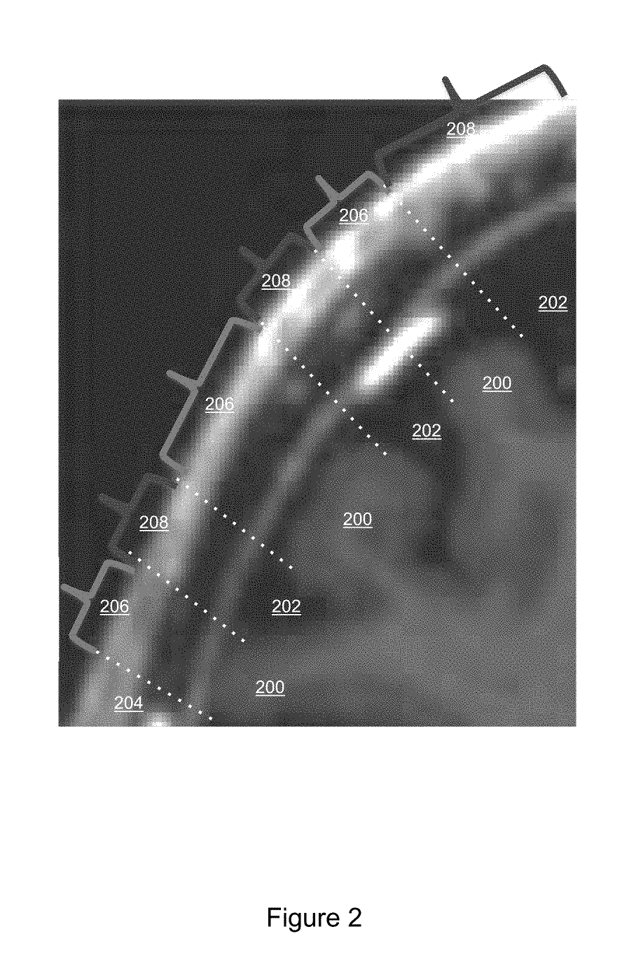

FIG. 2 illustrates aspects of the positioning of the EEG and MEG probes. Gyri 200 are part of a system of folds that create a larger surface area for the brain. Gyri are ridges that are generally adjacent to sulci 202 which are furrows. In certain areas of the brain the gyri are proximate to the calvarium 204, e.g. located immediately underneath to and abutting the inner table of the calvarium. Such locations 206 may be optimal for signal detection. In other areas of the brain, e.g. proximate to sulci, there is a pool of cerebrospinal fluid underneath the inner table of the calvarium. Such locations 208 are not optimal for signal detection because the signal must travel a longer distance through cerebrospinal fluid (CSF) before reaching the measurement probe. The current standard method of EEG probe placement involves placing the leads at positions relative to the scalp, not placement at positions relative to the locations of the gyms underneath the calvarium.

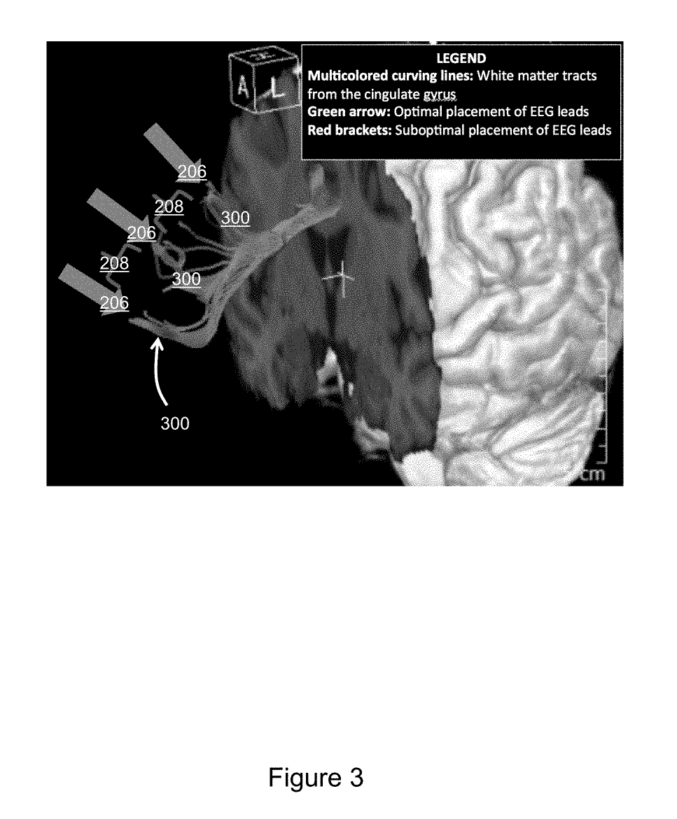

FIG. 3 is a volume rendered (VR) image of a DTI image superimposed on an isotropic T1 MRI image of the cingulate gyms. The optimal locations 206 and suboptimal locations 208 are shown with respect to axon tract clusters 300 that emerge from deep within the brain. It can be seen that there are gaps between the tract clusters 300.

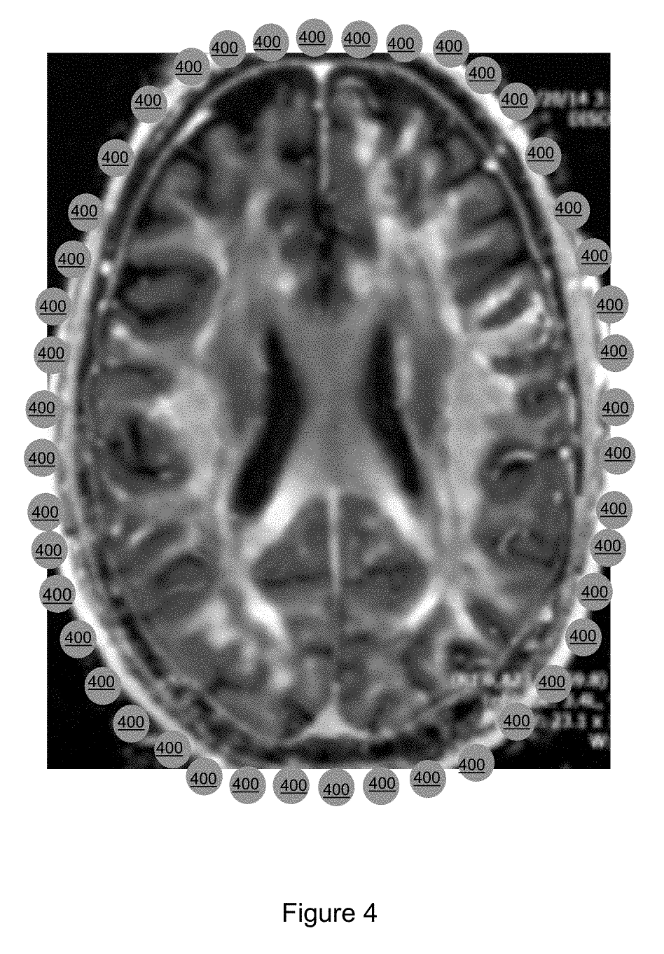

FIG. 4 illustrates EEG leads 400 superimposed on the DTI image. The EEG and DTI images are registered onto a common coordinate system. The colors in the DTI image represent the directions of the tract clusters, with green representing a tract that runs anteriorly-posteriorly, red representing a tract that runs transversely and blue representing a tract that runs superiorly-inferiorly.

Referring to FIGS. 2 through 4, an aspect of the presently disclosed technique may include placement of EEG probes at locations 206 that are optimal for signal detection as indicated by the isotropic sequence and DTI imaging that indicates the emerging white matter tracts into the gyri. Anatomically enhanced EEG positions may be placed via gyral anatomy, particular Brodmann areas or algorithms based on the proximity of the gyri to the skin surface on the scalp. In the event that there are metallic structures embedded in the scalp, calvarium or intracranially, revised positions would be established and recorded. The calculated or observed “ideal” positioning coordinates of the EEG electrodes could be determined and transferred to an EEG helmet system as will be described in greater detail below. The EEG leads would be placed accordingly. Fine tuning of the EEG lead placement could be performed based on the signal received at each location. Guided positioning of the EEG leads could take place during simultaneous EEG-MRI or after MRI is completed by using common reference points on the calvarium (e.g., eyes, ears, nose, or skin markers). The key point is that the DTI images and EEG images are placed on a common coordinate system, such that the signal received at the EEG can be correlated to the underlying brain structure. Guided-placement of EEG leads relative to tract clusters, and thus proximate to gyri, helps to optimize signal detection.

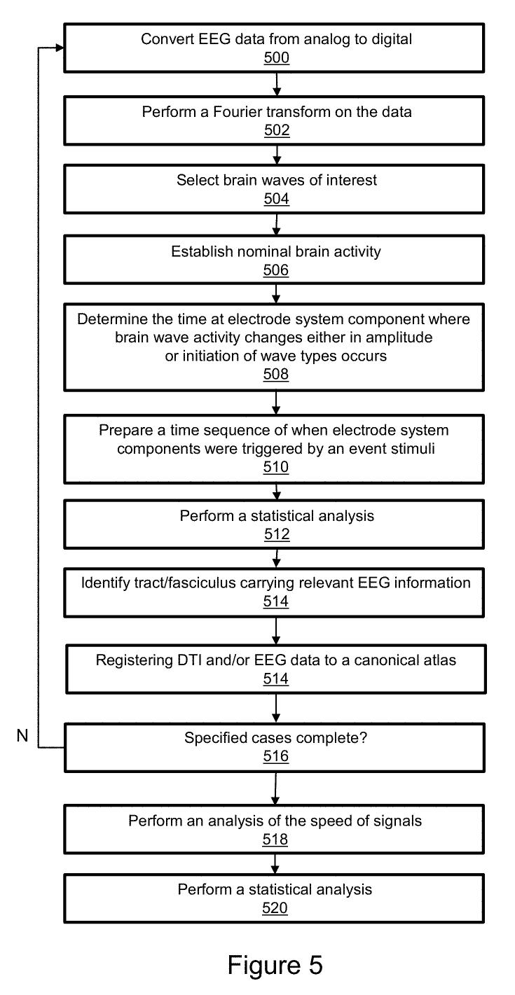

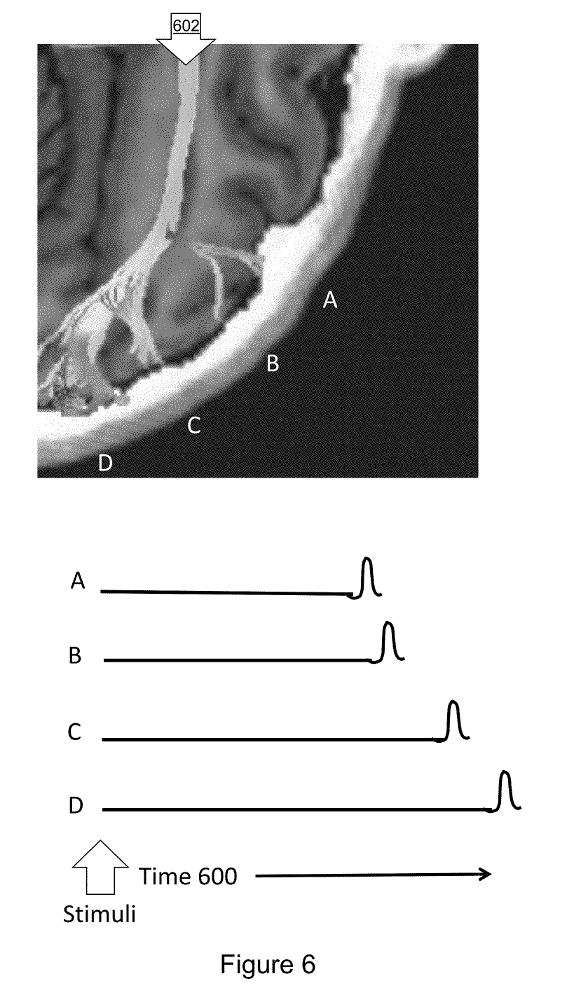

FIGS. 5 and 6 illustrate aspects of data processing and analysis. Subsequent to data collection, data analysis is performed. The purpose of this analysis process is to determine the speed at which the patient's brain operates and the sequencing and location of brain activity as it relates to the different types and location of stimuli. In essence, this process determines how the patient thinks. In step 500 the EEG analog data is converted to digital. Step 502 is to perform a Fourier transform on the data to separate the various brain waves. Step 504 is to select brain waves of interest. For example, Beta waves may be initiated when the point source of light is turned on. Gamma waves may reflect the thinking process for facial recognition. Step 506 is to establish nominal brain activity (i.e., resting state) during the first period of the examination during which no sensory stimuli are initiated. This would include determining brain wave frequencies, average and peak wave intensities, and presence (or absence) of Beta and Gamma waves. Step 508 is to determine the precise time 600 at electrode system components (e.g. A, B, C, D) wherein brain wave activity changes either in amplitude or initiation of wave types occurs. These activities would result from stimuli events closely timed and preceding the activity at each electrode system component. The response time between stimuli events and resultant brain activity at each of the electrode system components is calculated. Note that signal arrives at the scalp at EEG lead A, then at EEG lead B, then at EEG lead C, and then at EEG lead D in temporal sequence. The waveforms are all similar in shape, but in practice there may be variation because each gyms in the visual cortex corresponds to a different portion of the field of view and the waveforms received would vary based on the light detected in the eyes. Using the path lengths from the DTI images and the time of delays, the rate of neuronal conduction can be calculated. Step 510 is to prepare a time sequence of when the electrode system components were triggered by an event stimuli 602. Step 512 is to perform a statistical analysis (e.g., mean and standard deviation) of response times in cases where stimuli are replicated. In step 514 the tract/fasciculus carrying relevant EEG information is identified. This may be accomplished through correlational methods (e.g. phase-phase, magnitude-phase, magnitude-magnitude coupling) or coherence measures. Once the tract is identified, causality can be inferred (i.e., the origin of the traveling wave EEG signals along the DTI tract) by altering the ground or reference electrode for calculating voltage potential differences. A sign reversal, among other key changes in the EEG signature including its phase and magnitude, may be used to infer directionality (see Figure). This change in reverence/ground electrode can occur at the time of data collection or afterwards at the data analysis stage. Step 514 is registering DTI and/or EEG data to a canonical atlas that is built based on tract fiber lengths and/or thicknesses in their relationship to EEG temporal and/or frequency content, their direction, and/or their sources, origins, granular layer, connectivity, etc. Analysis continues by repeating the process of steps 508 through 514 for each case specified in the test procedure that covers each type and location of stimuli input as indicated by decision block 516. Step 518 is to perform an analysis of the speed of signals between electrode system components based on the sequence determined in step 510. Step 520 is to perform a statistical analysis (e.g., ANOVA) of cross stimuli response times and cross location of stimuli input locations to determine if statistical differences exist.

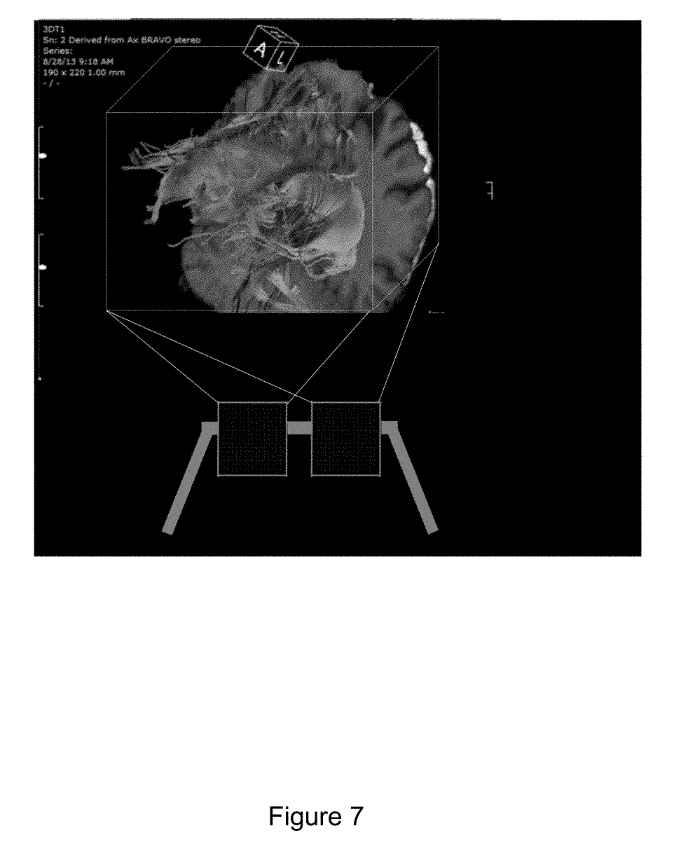

Referring to FIG. 7, which illustrates an overview of DTI, EEG and MEG on the same coordinate system, the EEG system electrode components have a high data readout, which will enable an understanding of timing and sequence of brain activity as activity is triggered by signals traversing the axons. Matching EEG and MEG to the DTI images allows creation of a “4D electrical conduction pathway through the brain.” E1 is the white lead anteriorly near Broca's region and E4 is the white lead posteriorly near superior Wernicke's region with E2 and E3 located in between E1 and E4. Similarly, the M1sa, M1sb, M1sc and M1sd surface detectors are located anteriorly around the E1 lead and near Broca's region. The M4sa, M4sb, M4sc and M4sd surface detectors are located posteriorly around the E4 lead near superior Wernicke's region. Surface MEG detectors M2sa, M2sb, M2sc, M2sd are located around the E2 lead. Surface MEG detectors M3sa, M3sb, M3sc, M3sd are located around the E3 lead. The M1R2, M2R2, M3R2 and M4R2 MEG detectors are on the concentric rings located just past the surface. The M1R3, M2R3, M3R3 and M4R3 detectors are on the concentric rings located just past the M1R2, M2R2, M3R2 and M4R2 MEG detectors. The M1R4, M2R4, M3R4 and M4R4 detectors are the outermost MEG detectors on the concentric rings located just past the M1R3, M2R3, M3R3 and M4R3 MEG detectors. Note that the firing begins in the left frontal region near Broca's region and at the initial time point there is signal seen at the E1 electrode. Its pathway is initially on white matter tracts that are oriented mostly transversely and are therefore color coded red to correspond to the location along the DTI anisotropy map. Its pathway becomes orange, yellow and then green as its white matter tract changes direction to a more anterior-posterior orientation. Then its pathway turns yellow, then orange then red as it becomes more transversely oriented. Note the changes in electric and magnetic signal over time. At the initial time point, there is both electric and magnetic signal at the surface EEG lead (E1) and MEG detectors (M1sa, M1sb, M1sc, M1sd, M1R2, M1R3 and M1R4). As the neuronal tract is carrying signal deeper and more posteriorly in the brain from the transition point from the red to orange to green region, there is no signal observed at the surface EEG lead (E2), but there is signal observed at the MEG leads (M2sa, M2sb, M2sc, M2sd, M1R2, M1R3 and M1R4) through electromagnetic induction. As the neuronal tract is carrying signal more superficial and more posteriorly in the brain from the transition point from the green to yellow to orange to red region, there is no signal observed at the surface EEG lead (E3), but there is signal observed at the MEG leads (M3sa, M3sb, M3sc, M3sd, M3R2, M3R3 and M3R4). At the final time point, there is both electric and magnetic signal at the surface EEG lead (E4) and MEG leads (M4sa, M4sb, M4sc, M4sd, M4R2, M4R3 and M4R4). This pathway would represent one firing connection and could be summarized by the time and particular named fiber tract activated. A series of times and associated fiber tracts activated would be used to develop the “4D electrical conduction pathway through the brain.” The MEG data may be used in concert with the EEG signal as an aid in spatially locating the electrical signal through the brain.

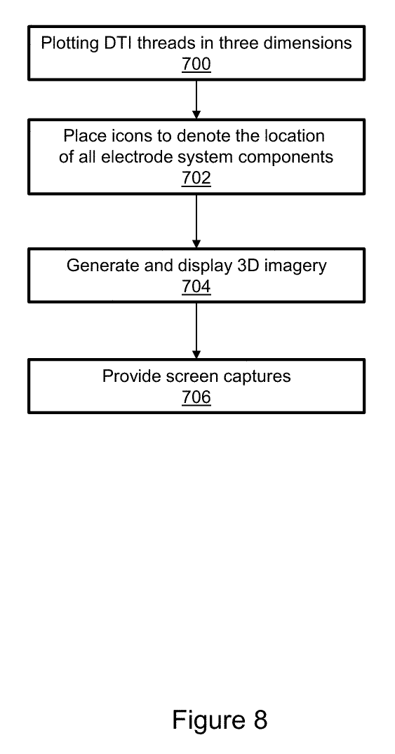

FIG. 8 illustrates a process to visualize the DTI results, place electrode system components and time sequence brain activity recorded by the system electrode components. Step 700 is plotting in three dimensions all DTI threads. Color coding of the voxels of thread groups may be applied to denote connectivity direction of different groupings of threads (e.g., from the frontal portion of brain to rear portion). Step 702 is, using augmented reality, placing icons to denote the location of all electrode system components. Other icons may be used to denote the direction of flow along the DTI threads. Computational information may be added such as time delays between sequential system electrode components. Step 704 is to generate and display 3D imagery to physician. This may be implemented in accordance with U.S. Pat. Nos. 8,384,771; 9,349,183; and published U.S. patent application 2016/0026266, each of which is incorporated by reference. Step 706 is using selected time sequencing of electrode system component activity (per data analysis) to provide a series of screen captures showing a sub-set of DTI threads and the current and previous (or next) electrode system component which had activity. Variations regarding which electrode system components would be highlighted could be made at discretion of physician.

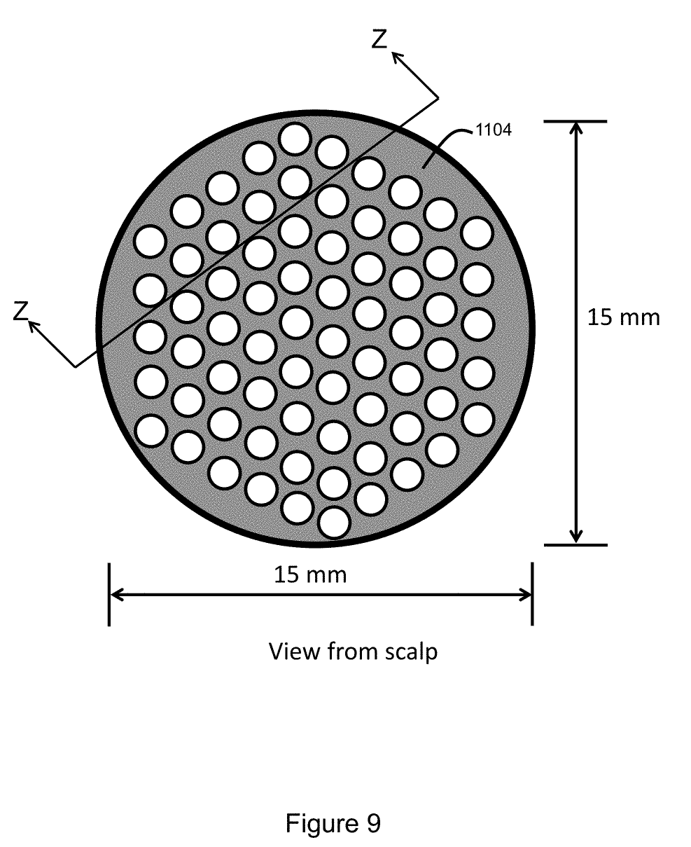

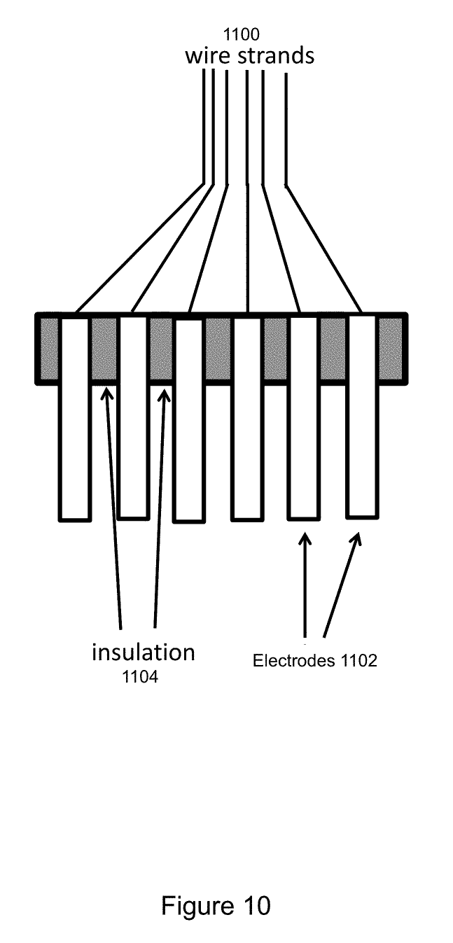

FIGS. 9 and 10 illustrate an example of a high density multiple electrode EEG probe for use in the technique described above. In contrast with current single wire/single electrode probes, the illustrated probe includes multiple wire strands 1100 emanating from each of multiple electrodes 1102 separated by insulation 1104 covering approximately the same surface area as prior art probe designs (10-20 mm in diameter). The axons (or tracks) that pass signals between different regions of gray matter tend to approach the scalp in clusters. A cluster may include one thousand or more tracks, and clusters are not evenly spaced around the scalp. The illustrated design helps to place multiple electrodes in position for signal detection with respect to a cluster region. Initial electrode density could be, but is not limited to, approximately 30 electrodes on a 15 mm diameter probe component. This density could increase to 1,000+ electrodes per probe component. Sampling rates will initially be 20 kHz, but may increase based on analysis of measured data. Instantaneous voltage in the area of the electrode may be amplified, and differential voltage output from pairs of electrodes may be plotted over time. Reference electrodes are also part of the system and can be implemented with a single electrode.

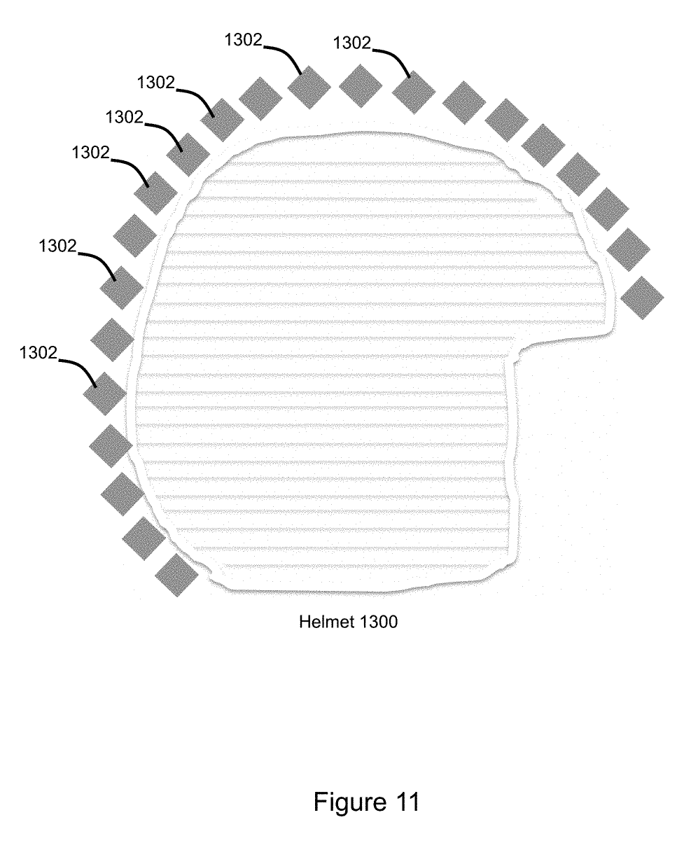

Referring now to FIG. 11, a helmet 1300 with an array of high-density multiple electrode EEG probes 1302 may include 90 or more probes nominally positioned in accordance with DTI connectivity. The helmet system may expand and contract to correspond to the size and shape each particular patient's head. The probes may be manually positioned. Alternatively or additionally, each probe component may be programmed to move to specific coordinates based on DTI and the structural neuroimaging map (e.g. 1 mm isotropic T1 MRI sequence). Whether manually, automatically or both, each probe component may be positioned over the center of a track cluster. A nominal starting point for the number of electrode system components would be 90+ generally relating to BrainSuite connectivity (http://brainsuite.org/visualization/connectivity/). The majority of current EEG systems use a net with electrodes roughly evenly spaced. The exact position of any particular electrode is not calculated and can change over time if the patient's head moves or if the net slips. There is only a general relationship between the placement of electrodes and the patient's anatomy as provided in the International 10-20 System.

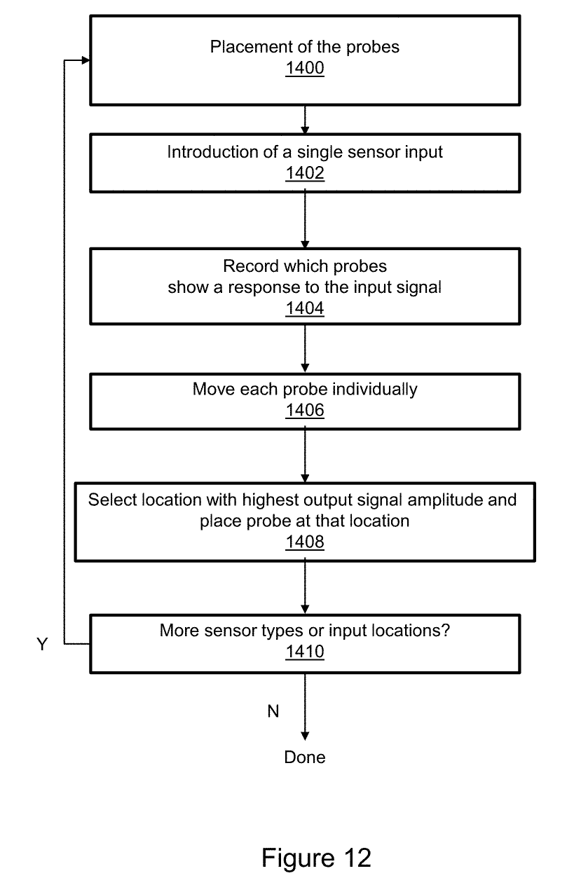

FIG. 12 illustrates a calibration process for fine-tuning the positioning of EEG probes. The process includes sensory inputs (e.g., point light source, sound, tactile input such as buzz on finger) and small movements of the probes such output signal response will be optimized. Step 1400 is placement of the probes at locations specified in accordance with the DTI. Step 1402 is introduction of a single sensor input (e.g., point light source into right eye). Step 1404 is to record which probes show a response to the input signal. Step 1406 is to individually move each probe which has shown a response to the sensor input, e.g. in directions up/down and right/left and combinations thereof sequentially and record output signal amplitude. Step 1408 is to select the location with highest output signal amplitude and place the probe at that location. Steps 1400 through 1408 are repeated with each different sensory input type and location as indicated in block 1410.

A number of features, aspects, embodiments and implementations have been described. Nevertheless, it will be understood that a wide variety of modifications and combinations may be made without departing from the scope of the inventive concepts described herein. Accordingly, those modifications and combinations are within the scope of the following claims.

What is claimed is:

1. A method of characterizing neurological function of a brain of an individual, comprising:
performing an MRI scan of the brain that includes a volumetric isotropic sequence and a diffusion tensor imaging (DTI) sequence to generate connectivity maps;
establishing a common coordinate system for the connectivity maps and external features of the individual;
positioning electroencephalography (EEG) probes based on the connectivity maps using the common coordinate system;
performing an EEG scan during performance of prescribed tasks to generate EEG images; and
generating 4D maps of neuronal activity from the EEG images and the connectivity maps.

2. The method of claim 1 comprising positioning magnetoencephalography (MEG) probes based on the connectivity maps using the common coordinate system.

3. The method of claim 2 comprising performing a MEG scan during performance of prescribed tasks to generate MEG images.

4. The method of claim 3 comprising generating the 4D maps of neuronal activity from the MEG images, the EEG images and the connectivity maps.

5. The method of claim 1 wherein the brain is within a skull, and wherein using the connectivity maps to position the EEG probes comprises positioning individual EEG probes proximate to locations where clusters of tracts are in close proximity to the skull.

6. The method of claim 1 comprising generating 4D maps of neuronal activity at a later time, thereby providing temporally distinct 4D maps of neuronal activity.

7. The method of claim 6 comprising performing a comparison of the temporally distinct 4D maps of neuronal activity.

8. The method of claim 1 comprising performing calibration before performing the EEG scan during performance of the prescribed tasks, the calibration comprising adjusting at least some EEG probe positions based on brain response to single sensor input.

9. The method of claim 1 comprising performing calibration before performing the EEG scan during performance of the prescribed tasks, the calibration comprising performing an EEG scan during a quiet period to document resting state brain activity.

10. The method of claim 9 wherein the calibration comprises performing an EEG scan after the quiet period and while administering a series of stimuli.

11. The method of claim 1 comprising analyzing neuronal activity by converting EEG data to digital form and performing a Fourier transform on the digital EEG data to separate different brain waves.

12. The method of claim 11 comprising establishing nominal brain activity based on EEG data collected when no sensory stimuli are initiated.

13. The method of claim 11 comprising determining response time to input stimuli based on changes in brain wave activity at the EEG probes.

14. The method of claim 13 comprising preparing a time sequence of when EEG probes were triggered by the stimuli.

15. The method of claim 14 comprising performing a statistical analysis of response times where the stimuli are replicated.

16. The method of claim 11 comprising identifying a tract through correlation or coherence with the stimuli.

17. The method of claim 16 comprising inferring causality with respect to the identified tract by altering a ground or reference electrode for calculating voltage potential differences.

18. The method of claim 11 comprising registering DTI and EEG data to a canonical atlas that is built based on tract fiber lengths and thicknesses in relationship to EEG temporal and frequency content, direction, and sources.

19. The method of claim 11 comprising performing an analysis of speed of signals between EEG probes.

20. The method of claim 11 comprising performing a statistical analysis of cross stimuli response times and cross location of stimuli input locations.

21. The method of claim 11 comprising plotting in three dimensions all DTI threads and color coding voxels of thread groups.

22. The method of claim 11 comprising, using augmented reality, placing icons to denote locations of the EEG probes.

23. The method of claim 22 comprising representing direction of flow along the DTI threads, and time delays between sequential EEG probes.

24. An apparatus comprising:
a plurality of signal electrodes disposed in an array with insulating material between ones of the electrodes; and
a reference electrode, wherein electrical output from each of the signal electrodes is measured relative to the reference electrode.

25. The apparatus of claim 24 wherein the array comprises 30 electrodes and the insulating material is circular with a 15 mm diameter.

26. The apparatus of claim 24 wherein the array comprises at least 1000 electrodes.

27. An apparatus comprising:
a helmet comprising:
a net; and
movable electroencephalography (EEG) probes.

28. The apparatus of claim 27 wherein the net conforms to a patient's head shape.

29. The apparatus of claim 27 comprising an automated system that repositions the movable EEG probes based on a diffusion tensor imaging (DTI) scan.
